(19) United States
(12) Patent Application Publication
(10) Pub. No.: US 2009/0300829 A1
(43) Pub. Date: Dec. 10, 2009

(54) Face Mask with Air Flow Means

(76) Inventor: Adam Matthew Spielvogel, The Woodlands, TX (US)

Correspondence Address: Bruce Stauffer, 39 N Brokenfern Dr., The Woodlands, TX, 77380, US

(21) Appl. No.: 12/134,234

(22) Filed: Jun. 6, 2008

(51) Int. Cl. A61F 9/02

(52) U.S. Cl. 2/438

(57) ABSTRACT

A face mask for use with Self Contained Underwater Breathing Apparatus (SCUBA) diving equipment which forms a sealed enclosure about the user's eyes and nose but not the mouth has a frame with sides and a clear viewing port and a means to introduce air flow into the enclosure. The flow of air into the enclosure is metered by a metering device which may be an orifice or a manually operated valve or an automated valve using a timer or sensors which monitor and respond to conditions such as heat and humidity. The metering device may be controlled by a control means. When the control means is activated, pressurized air flows into the enclosure. In one embodiment, air flow is directed against the inner surface of the viewing port to remove moisture and heat which may cause the viewing port to fog. The mask may be equipped with a relief valve to allow incoming pressurized air flow to force trapped liquid out of the enclosure.

### FIELD OF THE INVENTION

The present invention generally relates to the field of protective face masks and more particularly to face masks used with SCUBA equipment.

### BACKGROUND OF THE INVENTION

There are many types of face masks in the art. There are protective face masks for sports such as paintball and snow skiing. There are respirator masks for fire and dangerous environment safety. There are surgical masks, underwater snorkeling masks, racecar, air plane pilot and deep sea commercial diving masks and helmets. All of the mask and helmet designs identified as related art herein incorporate a clear viewing port to enable the user to see out of the mask. Many of these masks have design elements intended to alleviate the ubiquitous problem of viewing port fogging caused by humidity released by the user's exhaled breath and heat given off from the skin which limits vision of the user and thereby limits the utility of the face mask. Fogging of the viewing port is a problem endemic to face masks of all types and there have been many ways proposed to deal with this problem.

The simplest method of dealing with fogging of the interior of the viewing port is a simple wiping device as in U.S. Pat. No. 6,221,170B1, or the use of a small reservoir of water to slosh against the interior of the viewing port as in U.S. Pat. No. 6,115,848A. Another simple method introduced here by way of demonstrating the wide nature of the problem and the many attempts to combat this situation is found in U.S. Pat. No. 4,996,981A which teaches a heated viewing port lens. More complex methods attempt to inhibit fogging of the viewing port by creating a separate compartment for the eyes and another for the nose and mouth. Other designs incorporate air flow generating devices to help dispel moisture laden air which may accumulate in the eye compartment. These devices range from simple ventilation ports for air cross flow during sporting activity such as in U.S. Pat. No. 4,764,990A and U.S. Pat. No. 4,989,274A to more complex arrangements equipped with a de-fogging fan such as EP1506758A1. Other face masks utilize the user's intake of breath as the means for drawing fresh air into and through the eye area such as in U.S. Pat. No. 2,891,541A and U.S. Pat. No. 6,920,880B1.

Often face masks are used in snorkeling and other water sports. These have similar viewing port fogging problems with the additional problems of sealing the user's face against ingress of water and providing a means to allow access to air. These designs use some of the same devices as seen in the previously referenced art such as separate chambers to direct exhaled air away from the eye area such as US20010013346A1. The method of utilizing user's intake of breath as the means for drawing fresh air into and through the eye area is seen in U.S. Pat. No. 3,721,236A.

A face mask for use in a fire situation is disclosed in U.S. Pat. No. 5,687,713A. In the full face embodiment, breathing air is directed against the interior of the viewing port as it is released from the self contained air supply into the sealed face area. Another embodiment simply isolates the eye compartment from the breathing area to reduce fogging.

Other applications which require de-fogging of the viewing port are seen in full-head gear such as racing, aircraft, and commercial diving helmets. U.S. Pat. No. 3,293,659A and U.S. Pat. No. 3,362,403A disclose high altitude pilot's full head helmets with cushioning means. They both incorporate a means of directing incoming breathing air against the interior of the viewing port, either continuously, or by a demand regulator. A similar device for use while driving a race car is disclosed in U.S. Pat. No. 6,973,676B1. Breathing air is forced into the helmet and is directed against the interior of the viewing port. U.S. Pat. No. 5,704,073A discloses a pilot's breathing apparatus covering the mouth and nose, but with a quickly attachable face mask portion which when in place receives air directly from the existing breathing apparatus covering the mouth which air is directed against the interior of the viewing port upon entry onto the viewing port portion of the apparatus.

Deep sea commercial diving helmets also have the problem of viewing port fogging and similar methods are used to dispel fog from the interior of the viewing port. U.S. Pat. No. 3,995,627A provides for a diving helmet that can adapt to different types of gas input devices which gas is directed against the interior of the viewing port upon entry onto the viewing port portion of the apparatus. U.S. Pat. No. 3,680,556A similarly directs the primary air supply against the interior of the viewing port upon entry and has additionally a secondary safety air supply tank which has an on-demand valve which can be used for additional de-fogging or emergency air pressure also directed against the interior of the viewing port upon entry.

From a review of the above references it is clear that face mask fogging is an important issue in the design of a face mask or helmet. Many devices have been employed to solve this problem and enhance the utility of the referenced inventions. Some of the referenced designs incorporate separate chambers for the eyes and for the nose and mouth to separate exhaled air from the viewing port area. This technique of separating the user's nose and mouth from the user's eyes with separate compartments and thereby blocking exhaled air from the eye compartment cannot be used with SCUBA equipment since SCUBA equipment provides air directly to the user's mouth via a regulated high pressure air supply. The user's nose must be in the same enclosure as his eyes when using standard SCUBA equipment. Other referenced designs require the user's action of breathing to draw air past the viewing port for de-fogging. These ideas are impractical for the SCUBA application because the standard SCUBA breathing system requires breathing through the mouth via a standard regulator system which system cannot be adapted to pass air through the mask area before becoming available for breathing.

Diving masks disclosed in the related art teach commercial deep sea diving designs in which the user's entire head or face is covered with one enclosure. Breathing air is introduced into that enclosure being dispensed against the interior of the viewing port, performing a defogging function. In these designs, the same air dispensed for de-fogging means is used for breathing, which air enters the helmet, impinges on the interior of the clear viewing port, and then is subsequently breathed by the user. In a SCUBA application, the same air used for de-fogging the face mask cannot be commingled with the air flow used for breathing because of the design of the typical SCUBA breathing mouthpiece which requires all breathing air to enter the user's mouth from a regulated source. The SCUBA application requires that only the eyes and nose be sealingly enclosed in the SCUBA mask, effectively separating the air inside the mask from the air breathed through the breathing regulator.

None of the referenced designs are directed toward the needs of recreational SCUBA equipment users. With present equipment available to SCUBA divers, the most common way to defog a mask is to manually allow a little water into the mask, move it around the interior of the viewing port, and then blow the water out of the mask with air from the user's nose. This technique requires substantial training to be used effectively, and only experienced users have mastered this technique. Therefore it would be advantageous to provide a practical de-fogging and de-watering face mask that can be used with SCUBA equipment.

### SUMMARY OF THE INVENTION

The present invention consists of a face mask for use with SCUBA diving equipment having a frame with sides and a clear viewing port forming a sealed enclosure about the user's eyes and nose but not his mouth further consisting of a means to introduce air flow into the enclosure such as a hose coming from the SCUBA air supply. The air flow coming through the means to introduce air flow is metered by a metering device which may be an orifice or a manually or automatically operated valve. In one embodiment, the metering device is controlled by a control means. When the control means is activated, the metering device is allowed to start the flow of pressurized air into the enclosure. Said control means may be situated anywhere on the mask, regardless of the location of said means to introduce air flow or said metering device.

The frame of a SCUBA diving mask is usually made of a rigid material such as plastic which provides support for the viewing port and the skirt. The skirt is a flexible part attached to the frame which provides sealing action when worn snuggly against the user's face and generally seals around the user's eyes and nose. Since there are many combinations and types of SCUBA diving masks available, this disclosure refers to any combination of a mask's frame and flexible skirt as the mask's “sides”. In another embodiment, said means to introduce air flow is situated penetrating said side and the air flow is directed against the inner surface of the viewing port to remove moisture and heat which may cause the viewing port to fog.

Standard SCUBA equipment typically uses an air tank or cylinder containing compressed air at about 3000 pounds per square inch (PSI). Air pressure is reduced to an intermediate pressure of about 140 PSI by a first stage pressure regulator which attaches to the cylinder valve. The air for breathing is further reduced to ambient (surrounding) pressure by a second stage regulator which is attached to the divers breathing mouth piece. A typical first stage regulator provides several outlets for various uses such as supplying air to a buoyancy control device (BCD). Said means to introduce air flow would typically receive air from one of these outlets. However, introducing air flow into said enclosure at about 140 PSI could have unwanted effects. For instance, it could cause excessive use of air, the possibility of blowing the mask off the user's face, and excessive splashing of water into the user's face and discomfort of excessive air flow against the user's eyes and face. Therefore, another embodiment would include a pressure reducing means to reduce the line pressure of the air entering the mask into the range of 10 to 80 PSI. Said pressure reducing means could be located attached to the first stage regulator, or could be built into said metering device, or be located anywhere along the path of air flow from the cylinder to said enclosure.

In another embodiment, the air flow coming into the mask is directed against the interior of the clear viewing port by an air dispensing means. The air dispensing means which guides and disperses the incoming air against the viewing port may be a tube situated across the top of the viewing port with multiple exit ports which ports are oriented towards said interior of the clear viewing port so the flow of air will be smooth and evenly distributed across the interior of the clear viewing port. The air dispensing means may be a separate part or may consist of conduits built into the frame or skirt of the mask either partially or completely utilizing the perimeter of the mask, especially the area of the perimeter of the mask where the frame contacts the material of the clear viewing port.

In another embodiment, the mask may have a valve situated penetrating the bottom of said enclosure which allows water and/or air to be vented from the inside of the enclosure to the outside using said air flow under pressure.

In another embodiment said control means is activated by sensors which determine various conditions existing inside said enclosure such as temperature and humidity. When a temperature or humidity situation is detected by the sensors in excess of a predetermined setting, said control means activates the metering device which allows air to flow into said enclosure until said conditions are back in the predetermined acceptable range when said controlling means will deactivate the metering device stopping the air flow.

### BRIEF DESCRIPTION OF THE DRAWINGS

For a better understanding of the present invention, reference is made to the following detailed description and accompanying drawings wherein,

FIG. 1 shows a general view of the preferred embodiment of the inventive face mask as attached to the SCUBA air cylinder with the mask's sides and clear viewing port forming a sealed enclosure about the user's eyes and nose (user's head and face not shown), a means to introduce air flow into the enclosure, a means to meter the flow of air, a means to control the metering device, a means for dispensing and directing the air flow against the interior of the clear viewing port, a water escape valve exiting the bottom of the mask frame, and a strap for holding the mask sealingly to the user's face. Also shown is the typical SCUBA arrangement of the cylinder, the cylinder main valve, the first and second stage regulators, and additional outlets from the first stage regulator including the means to introduce air flow into said enclosure.

FIGS. 2-4 show various embodiments of the means for dispensing and directing the air flow against the interior of the clear viewing port using different methods to incorporate the air tubes or built in air conduits into the structure of the face mask, especially in the area where the side contacts or supports the clear viewing port.

FIGS. 5-8 show alternate placements of the control means for controlling said metering device, which control means may be placed anywhere on the mask, but preferably on the right or left side of the frame formed integrally with said metering device.

### DETAILED DESCRIPTION OF THE DRAWINGS

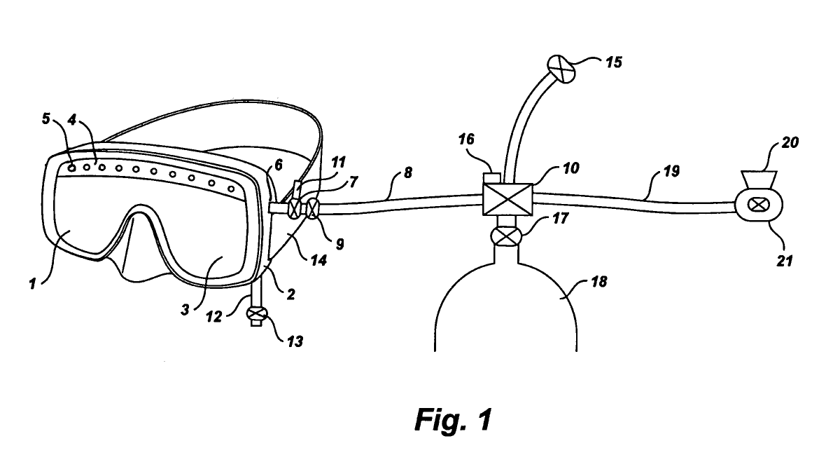

FIG. 1 shows the preferred embodiment of the present invention wherein the inventive face mask has sides 2 which in combination with the clear viewing port 1 create an enclosure 3 which sealingly encloses the user's eyes and nose but not his mouth (user's face not shown) being held in place by a strap 14. The means to introduce air flow 8 penetrates the side through penetration 6 and is metered by a metering device 7 controlled by a control means 11. Air for said means to introduce air flow is fed by air from the SCUBA tank 18 through cylinder main valve 17 and first stage pressure regulator 10 with an extra port for other pressurized air using devices 16 and a pressure reducing device 9. An air dispensing means 4, which in the preferred embodiment is a tube stretching between the opposite sides of the interior of the enclosure above the line of sight of the user, receives said air flow and dispenses it through multiple exit ports 5 which ports are oriented towards the interior of the clear viewing port. A means for allowing water to escape from the enclosure 13 penetrates the underside of the mask at penetration 12. Also shown is a hose 19 going from the first stage pressure regulator to the second stage pressure regulator 21 and mouth piece 20.

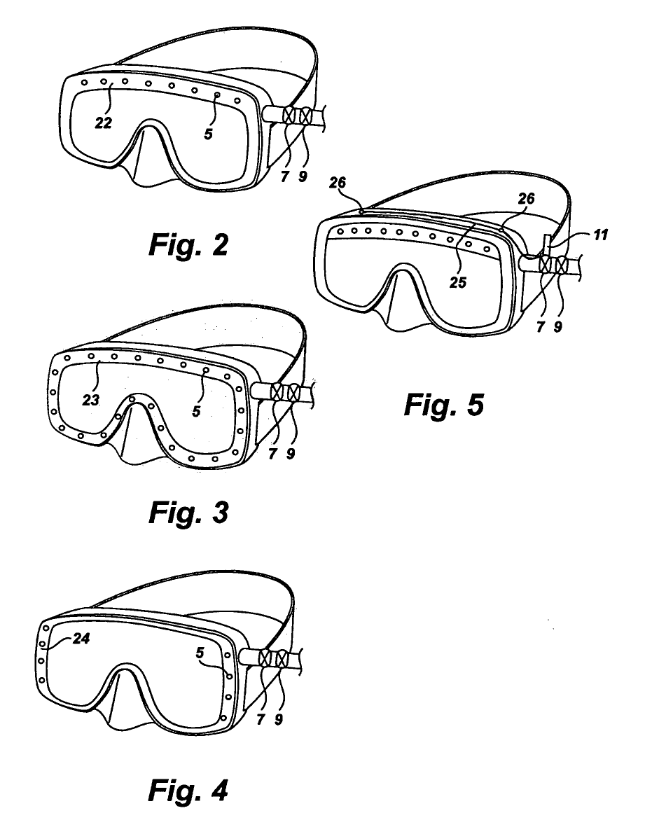

FIGS. 2-4 show various embodiments of the air dispensing means 5 using different methods to incorporate it into the mask's design. Referring now to FIG. 2, the air dispensing means 5 is a conduit built into the frame or side along the top of the area in close contact with the clear viewing port having multiple air exit ports pointing towards the interior side of the clear viewing port. In this way there is no extra equipment taking up limited space inside the mask enclosure.

Referring now to FIG. 3, the air dispensing means 5 is a conduit built into the frame or side along the entire area in close contact with the clear viewing port having multiple air exit ports pointing towards the interior side of the clear viewing port. In this way there is lower air flow from each port offering more comfortable and quiet operation.

Referring now to FIG. 4, the air dispensing means 5 is a conduit built into the frame or side along each side of the area in close contact with the clear viewing port having multiple air exit ports pointing towards the interior side of the clear viewing port. In this way there is better viewing area since the air conduits are moved to either side of the viewing area.

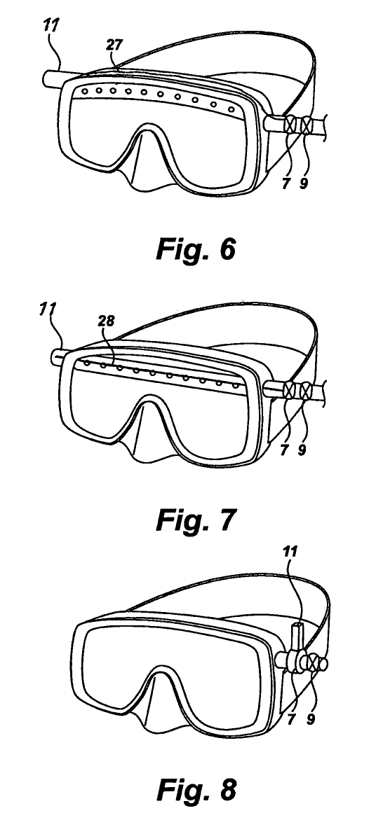

Referring now to FIG. 5, the metering device 7 is controlled by a control means 11 said control means activated by sensors 26 which determine various conditions existing inside said enclosure such as temperature and humidity. When a temperature or humidity situation is detected by the sensors in excess of a predetermined setting, said control means allows air to flow into said enclosure until said conditions are back in the predetermined acceptable range when said control means will stop the air flow. Said sensors may be connected by means of electrical conductive devices such as wires 25.

Referring now to FIG. 6, the control means 11 may be placed on the side of the inventive mask opposite the metering device 7 and operate the metering device remotely by electronic or other remote operating means, in one embodiment connected by a wire 27.

Referring now to FIG. 7, the control means 11 may be placed on the same side as the metering device 7 and integral with the metering device and operate the metering device directly manually or automatically.

Referring now to FIG. 8, the control means 11 may be placed on the side of the inventive mask opposite the metering device 7, operating the metering device remotely by a mechanical link 28.

In any embodiment, the means to introduce air flow into the enclosure, the metering device, and the control means may be placed in any location on the inventive mask. It will be appreciated by one of average skill in the art that the inventive idea of the present invention may be expressed in embodiments not explicitly set out herein without departing from the spirit of the invention.

1-14. (canceled)

15. An underwater diving face mask adapted for use with SCUBA equipment which forms a sealed enclosure about the eyes and nose but not the mouth of the user comprising a clear viewing port and a means for introducing a flow of pressurized air into said enclosure.

16. The face mask of claim 1 further comprising a means for metering said flow of air.

17. The face mask of claim 2 in which the means for metering said flow of air is an orifice.

18. The face mask of claim 2 further comprising a control means which controls said metering means.

19. The face mask of claim 4 in which the control means is manually operated.

20. The face mask of claim 4 in which the control means is operated by a timer.

21. The face mask of claim 4 in which the control means is operated by a sensor.

22. The face mask of claim 7 in which the control means is operated by multiple sensors.

23. The face mask of any of the above claims in which said flow of air impinges upon the interior surface of said viewing port.

24. The face mask as in any of claims 1-8 where said means for introducing pressurized air receives said air from the SCUBA air supply via a pressure reducing means.

25. A protective face mask comprising sides and a clear viewing port forming an enclosure about the user's eyes and nose but not the mouth having a means for introducing air into the enclosure from a pressurized air supply.

26. The mask of claim 11 in which said pressurized air is metered into said enclosure with a metering device.

27. The mask of claim 12 in which said metering device is controlled by a controlling device.

28. A method of defogging a diving mask for use with SCUBA equipment which comprises the steps of bringing air at a reduced pressure from the SCUBA air supply into the inventive diving mask and venting said air upon the interior surface of the viewing port of said mask.
